(19) United States
(12) Patent Application Publication
(10) Pub. No.: US 2021/0402171 A1
(43) Pub. Date: Dec. 30, 2021

(54) APPARATUS, CONTROL DEVICE, KIT FOR SUPPORTING THE HEART ACTION, INSERTION SYSTEM, AND METHOD

(71) Applicant: ARTRACT MEDICAL GMBH, Regensburg (DE)

(72) Inventor: Wolfgang GOETZ, Regensburg (DE)

(21) Appl. No.: 17/279,463

(22) PCT Filed: Sep. 26, 2019
(86) PCT No.: PCT/EP2019/076017

§ 371 Date: Mar. 24, 2021

(30) Foreign Application Priority Data
Sep. 28, 2018 (DE) 10 2018 124 048.1

(51) Int. Cl. A61M 60/861; A61M 60/196; A61M 60/258; A61M 60/289; A61M 60/468; A61M 60/47; A61M 60/489

(52) U.S. Cl. CPC A61M 60/861; A61M 60/196; A61M 60/258; A61M 60/489; A61M 60/468; A61M 60/47; A61M 60/289

(57) ABSTRACT

The present invention relates to an apparatus (500) for supporting the heart action, preferably by displacing the heart base (110) and/or the aortic root (201), comprising at least a first anchor (501) and a pulling device or guiding device (502, 503, 732, 732a, 732b) for moving the first anchor (501), wherein the first anchor (501) is provided and designed for implantation in or on the heart base (110), the heart skeleton (120), the aortic root (201) and/or a structure in local proximity to the aortic root (210), and/or comprising at least one lifting drive (502, 503). The present invention further relates to a control device (901), an insertion system, a kit and a method for supporting the heart action.

The present invention relates to an apparatus and to a control device for supporting the heart action or activity. The present invention also relates to an insertion system, a kit and a method for supporting the heart action.

Known for the care of patients with heart failure are various methods and systems for heart support or support of the heart action. For example, there are left ventricular and/or right ventricular heart support systems that may be powered or driven pneumatically, hydraulically or electrically. The systems may be fully implanted, wherein the energy supply may be, for example, telemetric or cable-based.

It is the object of the present invention to provide a further apparatus and a further control device for supporting the heart action. In addition, it is an object to provide a further insertion system, a further kit and a method.

The object according to the present invention is achieved by the apparatus of claim 1, by the control device of claim 11, by the insertion system of claim 12, by the kit of claim 13 and by the method of claim 14.

The object according to the present invention is achieved by an apparatus for supporting the heart action, preferably by moving the heart base and/or the aortic root, comprising at least a first anchor and a pulling device or guiding device for moving the first anchor and/or comprising at least one lifting drive.

The apparatus according to the present invention is designed to displace the heart base and/or the aortic root at least temporarily.

The present invention also relates to a control device for driving a pulling device or guiding device of the apparatus according to the present invention. It further relates to an insertion system for the apparatus. The insertion system serves for implanting the apparatus into the patient. The insertion system encompasses one or more of the following components in any combination: insertion catheter, guiding catheter, guiding wire and delivery or supply catheter. In this, the insertion system may in several embodiments also comprise multiple exemplars of the respective catheter and/or of the guiding wire mentioned.

The invention also relates to a kit which encompasses an apparatus according to the present invention for supporting the heart action, a control device according to the present invention and/or an insertion system according to the present invention.

The present invention also relates to a method, in particular for supporting the heart action, preferably by displacing the heart base, the heart skeleton and/or the aortic root or for thereby supporting and/or for implanting an apparatus according to the present invention. The method encompasses the steps of providing an apparatus according to the present invention and optionally an insertion system according to the present invention, implanting a first anchor in or on the heart base, the heart skeleton, the aortic root and/or a structure in local proximity to the aortic root, in particular by the insertion system, implanting the pulling device or guiding device for moving the first anchor, in particular by the insertion system according to the present invention and connecting the first anchor and the pulling device or guiding device, in particular by the insertion system.

The method according to the invention is in particular not intended or prepared for implanting the first anchor through a lumen of the mitral valve.

In particular, the method according to the present invention is not intended or prepared for implanting the first anchor around a circumference or around a partial circumference of the mitral valve ring (mitral valve annulus).

The method according to the present invention may further encompass the steps of implanting a second anchor in or on the heart apex, a ligament, a rib, a sternum and/or a structure with local proximity to the heart apex, and connecting the first anchor and the second anchor, in particular by the insertion system.

Furthermore, the method according to the present invention may encompass the steps of providing a control unit according to the present invention and moving the pulling device or guiding device using the control unit in order to support the heart action.

The so-called heart skeleton is anatomically located in the immediate vicinity of the aortic root and may include or comprise collagen fiber rings, cartilage substance, the right and left trigone, and heart valve rings.

With the aid of the method according to the present invention, the heart action may advantageously be supported by shortening, which may be referred to as modulation, of the long heart axis.

The implantation of the first anchor in or on the heart base, the heart skeleton, the aortic root and/or a structure in local proximity to the aortic root may be carried out surgically or by the insertion system via a body vein and via the vena cava to the right atrium. The first anchor may be implanted surgically, minimally invasively and/or percutaneously in the area of the heart base and/or in the area of the interatrial septum.

The second anchor may be implanted surgically, minimally invasively and/or percutaneously in the area of the heart apex, in a rib or near a rib.

The first anchor and the second anchor may be connected by an active, shortening element. The active, shortening element may be a pulling device or guiding device.

With the aid of the method according to the present invention, the distance from the heart base and/or from the aortic root to the heart apex may be actively shortened, thus increasing the ejection volume of the heart. The distance may also be the long axis of the heart, which essentially corresponds to the length of the interventricular septum.

With the aid of the method according to the present invention, displacing or stretching the aortic root (ascending aorta) during the systole may be supported. As a result, the stroke of the aortic root, including a retraction of the aortic root in diastole, can advantageously be increased.

In the context of the present invention, a patient can be a human or an animal. A patient may be sick or healthy.

In all of the following statements, the use of the expression “may be” or “may have” and so on, is to be understood synonymously with “preferably is” or “preferably has,” and so on respectively, and is intended to illustrate embodiments according to the present invention.

Whenever numerical words are mentioned herein, the person skilled in the art shall recognize or understand them as indications of numerical lower limits. Unless it leads the person skilled in the art to an evident contradiction, the person skilled in the art shall comprehend for example the specification of “one” as encompassing “at least one”. This understanding is also equally encompassed by the present invention as the interpretation that a numerical word, for example, “one” may alternatively mean “exactly one”, wherever this is evidently technically possible for the person skilled in the art. Both understandings are encompassed by the present invention and apply to all numerical words used herein.

Unless otherwise stated below, proximal means towards the center of the body and distal means away from the center of the body.

Advantageous developments of the present invention are each subject-matter of the dependent claims and of embodiments.

Whenever an embodiment is mentioned herein, it is then to be understood as an exemplary, non-limiting embodiment according to the present invention.

Embodiments according to the present invention may comprise one or more of the features mentioned above and/or below in any combination, unless the skilled person recognizes such a combination as being technically impossible.

In several embodiments according to the present invention, the guiding device is, or comprises, a lifting drive. Therefore, the embodiments shown and described below for the lifting drive apply in the same way to the guiding device and vice versa.

In some embodiments according to the present invention, the pulling device or guiding device is preferably substantially a linear guiding device or comprises such a device.

In some embodiments according to the present invention, the first anchor has a V-shaped or U-shaped section. Alternatively, the first anchor is V-shaped or U-shaped. The first anchor is preferably connected to at least one linear guiding sleeve, more preferably to two linear guiding sleeves.

One linear guiding sleeve may be connected to one end of the first anchor, two linear guiding sleeves may be connected to the two ends of the first anchor.

The linear guiding sleeve is designed to guide the linear guiding device.

In some embodiments according to the present invention, the linear guiding device is, or comprises, a piston.

In some embodiments according to the present invention, the linear guiding device may be moved mechanically, hydraulically, pneumatically, electrically or magnetically. It may be designed accordingly for this purpose. A corresponding drive or connection for such a drive may be provided.

In some embodiments according to the present invention, a tension spring is arranged in at least one of the linear guiding sleeves between the linear guiding device and the first anchor.

In some embodiments according to the present invention, the connection between the first anchor and the linear guiding sleeve is, or comprises a plug connection, a clamping connection, a bayonet lock or another connection.

In some embodiments according to the present invention, the pulling device or guiding device is, or comprises, an elongated, flexible and/or tensile element, in particular a rope or a belt.

In some embodiments according to the present invention, the device is made of, or comprises, a metal, a plastic, and/or a composite material. The metal, plastic and/or composite material is preferably biocompatible.

In some embodiments, a lifting drive preferably comprises a longer and a shorter operating state. In this, the lifting drive may preferably be switched from the longer operating state to the shorter operating state and back again to the longer operating state; this is preferably done periodically, particularly preferably synchronously with the patient's heart action.

The lifting drive as an optional part of the apparatus according to the present invention may preferably be placed in a heart such that a variable force is exerted on a part of the heart. For example, the lifting drive may be used to exert force on the heart base, the force preferably being directed towards the heart apex.

In several embodiments, two or more lifting drives are used in the apparatus according to the present invention. In this, it may for example be provided to insert a lifting drive into the left ventricle and a further lifting drive into the right ventricle. In this, preferably one of the two lifting drives, starting from the right ventricle, penetrates the tricuspid valve with its first end, preferably directly at the base of the valve leaflet. With its second end, the lifting drive is attached to, or penetrates, the heart apex, and it is fastened, for example, to the sternum, to the sterno-pericardial ligament, or to a rib, for example via an anchor.

In several embodiments, the other of the two lifting drives is placed in the left ventricle and penetrates the mitral valve with its first end, preferably at the transition from its anterior to its posterior leaflet, in particular in the vicinity of the commissure between the anterior and posterior leaflet, particularly preferably directly on the base of the valve leaflet. The second end of the lifting drive being placed in the left ventricle is attached to, or penetrates, the heart apex and is fastened, for example, to the sternum, to the sterno-pericardial ligament or to a rib, for example via a second anchor.

In several embodiments, the lifting drives are connected to each other directly or indirectly at their first ends via the first anchor, which may be referred to as a connecting piece. For this purpose, the connecting piece is preferably located in the atria of the heart after the implantation and thereby penetrates the interatrial septum.

The lifting drives are preferably fastened in the heart such that a shortening of the lifting drives leads to a support of the heart action, in particular by displacing the heart base and/or the aortic root. A change in the length of the lifting drives such as shortening or lengthening may mean a displacement of a piston relative to a cylinder of the lifting drives. If, for example, the piston is pushed into the cylinder of the lifting drive, the total length is shortened (shortening of the lifting drive); when the piston is pulled out or guided out of the cylinder, the total length is increased (lengthening of the lifting drive). This similarly applies to the guiding device. Pushing or pulling a linear guiding device into a linear guiding sleeve leads to a shortening of the guiding device, guiding or pulling the linear guiding device out of the linear guiding sleeve leads to a lengthening of the guiding device.

In several embodiments, the apparatus according to the present invention may be implanted completely surgically via an open-heart surgery and may be designed accordingly. In some cases, the apparatus may be inserted via catheter access. Alternatively, a combination of an open-heart surgery and catheter-based procedure is also possible.

In several embodiments, the shortening of the lifting drive(s) may take place hydraulically, pneumatically and/or magnetically (for example by a linear motor or a linear actuator). For this purpose, the lifting drives may have their own drive. Alternatively, a force may be supplied to the lifting drives externally to shorten them, for example from a separately arranged control unit.

In several embodiments, one or more ropes are present within the lifting drives and/or within the connecting piece, which may preferably be used to transmit power to the lifting drives in order to shorten them. Preferably, a rope is arranged within the lifting drive as a cable pull. In this, one end of the rope is preferably attached to a second anchor on the heart apex, on the sterno-pericardial ligament or on a rib, or provided for this purpose, wherein the other end is preferably free and both lifting drives (which may be supported at their ends) may be shortened by pulling on the free end of the rope.

In several embodiments, each lifting drive comprises a piston and a cylinder, with the piston being able to slide into the cylinder when the lifting drive is shortened. The shortening and lengthening of the lifting drives may be caused, for example, by a pressure change in a liquid contained in the cylinders. The liquid may for example be supplied from an internal or external control unit.

In several embodiments, elastic elements, for example compression or tension springs, which for example elastically counteract a shortening or lengthening of the lifting drives are introduced into the cylinders.

The control unit (also referred to herein as the control device) may have and/or be connected to a power source. The control unit may further have sensors that enable or contribute to controlling the apparatus synchronously with the heart action.

In several embodiments, the apparatus according to the present invention comprises at least one lifting drive which displaces the heart base and/or the aortic root during the heart action. A displacement may mean a shifting, in particular a translational shifting. As a result, the long axis of the heart is shortened and the aortic root is stretched, preferably in part of the heart cycle. The lifting drive is preferably moved synchronously with the heart action on the basis of sensor data.

Preferably, the apparatus is designed for at least partial implantation into a heart. The apparatus is preferably designed such that it can be at least partially implanted in a human heart. In several embodiments, the apparatus is designed such that it can be connected to a rib and/or to the sternum at one section and to the heart, in particular the heart base, in a further or another section.

In several embodiments of the apparatus according to the present invention, said apparatus is designed such that several of these apparatuses may be located in the heart at the same time and may support the hearts action. In this case, preferably a plurality of the apparatuses is implanted in one heart.

In several embodiments, after the apparatus has been implanted, a first section of the apparatus is preferably located in the left ventricle and a further section is located in the right ventricle. In several embodiments, there is one apparatus at least partially in the left ventricle and a further apparatus is at least partially in the right ventricle. In this, the apparatuses are preferably designed such that they may connect the heart base to the heart apex after being implanted in the heart, wherein the length of the apparatuses is variable.

In several embodiments, the first anchor is designed such that when the apparatus is shortened after the implantation, the heart skeleton, the heart base and/or the aortic root is/are pulled towards the heart apex. In this, for example, stored energy, e.g. spring energy or electrical energy, is converted into kinetic energy.

In several embodiments, the apparatus comprises a second anchor for implantation in or on the heart apex, a ligament, a rib, a sternum, and/or a structure in local proximity to the heart apex.

In several embodiments, the first anchor for implantation in or on the heart base comprises a bracket which may be implanted such that it extends from the left into the right atrium and thereby penetrates e.g. the lower end of the interatrial septum between the mitral valve annulus and the tricuspid valve annulus. The bracket may for example be a section of the first anchor.

After the implantation, the bracket lies preferably above or on top of the heart skeleton, in particular above the right trigone.

During the entire heart cycle, preferably the position of the second anchor does not change or does not change significantly after the implantation. When the apparatus is shortened, the heart skeleton, the heart base and/or the aortic root is pulled in the direction of the heart apex.

In some embodiments, the apparatus has more than two anchors, in particular three, four or six anchors.

In several embodiments, the apparatus is designed to shorten during the systole and lengthen during the diastole.

Preferably, the apparatus is used at a length that matches or that is adapted to the length of the heart and/or the height of the patient.

In the diastole, the apparatus preferably has a length between 4 cm and 20 cm, particularly preferably between 7 cm and 16 cm.

In the systole, the apparatus preferably has a length between 4 cm and 16 cm, particularly preferably between 6 cm and 14 cm.

Preferably, the apparatus shortens in the systole compared to the diastole by between 3% and 40%, more preferably between 5% and 20%.

The stated information on lengths and shortening preferably relate to the section of the apparatus that is located between the first and the second anchor.

Preferably, the apparatus supports the natural motion of the heart base, the heart skeleton, and/or the aortic root and increases or enlarges the amplitude of this motion, whereby the stretching of the aortic root in systole and the resulting elastic retraction of the aortic root in diastole are intensified, whereby in turn the diastolic pumping effect of the ascending aorta is supported.

In several embodiments, the first anchor is an implant, in particular an intravascular stent, for placement in the aorta or an aortic valve prosthesis.

In several embodiments, the energy the apparatus uses to support heart action is tension energy of an elastic element of the apparatus.

In this case, the tension energy is preferably transferred by an elastic element, in particular by a spring. The force of the elastic element preferably compensates for the missing elastic restoring force of the aortic root.

In several embodiments, the force applied to the apparatus for supporting the heart action is transferred mechanically or magnetically from a power source via a connection unit. The apparatus comprises preferably a connection unit for transferring force to the part of the apparatus implanted in the heart. The force transfer may be carried out, for example, via a Bowden cable, pneumatically, hydraulically or magnetically.

The apparatus preferably comprises, or is connected to, an energy source. In several embodiments, the energy source may provide a force that is transferred to the portion of the apparatus that shortens in the systole. For this purpose, the energy source comprises an element that can convert stored energy into kinetic energy, for example a linear actuator or an electric motor that may be designed as a rotating motor or as a linear motor. The energy source may alternatively or additionally have a compressor for providing a fluid under pressure. For example, a battery may be used as energy storage device.

In several embodiments, the apparatus includes a unit for converting transferred energy from a separate energy source. In several embodiments, energy is provided to the apparatus in the form of electrical energy. The apparatus preferably has an element that can convert the electrical energy into kinetic energy, for example an electrical, rotating motor or linear motor or a linear actuator.

In several embodiments, the force used by the apparatus to support the heart action is a pneumatic or hydraulic force. For this purpose, a fluid is pressurized in a portion of the apparatus or outside of the apparatus, wherein the fluid is preferably in communication with a movable portion of the apparatus. The pressure of the fluid is preferably variable during a heart action. The apparatus preferably comprises a hydraulic or pneumatic cylinder for converting the pressure into a force for shortening and/or lengthening the apparatus.

The pneumatic force or hydraulic force is generated inside the apparatus and/or outside the apparatus. In several embodiments, electrical energy is transferred to the apparatus via an electrical conductor where it is converted to pneumatic and/or hydraulic energy.

In several embodiments, the apparatus encompasses an energy source that provides the energy needed to support the heart action as electrical, pneumatic, hydraulic, or magnetic energy.

Said forms of energy are preferably used to shorten and/or lengthen portions of the apparatus.

In several embodiments, the energy source is powered by nuclear energy.

In several embodiments, the apparatus is bistable between a diastolic up position and a systolic down position. This means that the apparatus is preferably arranged or positioned in a stable manner in a diastolic and/or a systolic end position, i.e., preferably does not move out of this position without external intervention.

In several embodiments, the apparatus has a state of equilibrium in its upward or downward position (or upper or lower position), wherein the apparatus preferably moves between the two stable positions during operation when energy is controllably supplied to the apparatus from one of the separate energy sources.

In several embodiments, the apparatus according to the present invention or the kit according to the present invention comprises a control device. A control device is also referred to herein as a control unit, and vice versa. The control unit is thereby preferably configured to control the device depending on the heart cycle. Preferably, the control unit comprises a computing unit.

In several embodiments, the control unit comprises, or is connected to, sensors. The sensors are thereby preferably suitable for measuring physiological parameters associated with cardiac activity. The sensors are preferably designed such that they provide a sensor signal that is made available to the control unit. The control unit is thereby configured to control or regulate the shortening and/or tensile force of the apparatus, preferably depending on sensor signals.

In several embodiments, the control unit controls the frequency, speed, pauses, and/or force of the repetitive shortening and lengthening of the apparatus.

In several embodiments, the control device encompasses, or is connected to, sensors that measure electrical cardiac activity. In several embodiments, the sensors encompass pressure sensors (which, for example, measure the blood pressure in vessels and/or in one or more ventricle(s), sensors for force, electrical voltage and/or electrical current and/or sensors for the extent to which the apparatus is shortened. In several embodiments, the control device records an EKG signal or another signal in order to control the apparatus synchronously with the heart action.

In several embodiments, the apparatus according to the present invention may be implanted with the aid of cardiac surgery, minimally invasive surgery or with the aid of a percutaneous catheter, wherein the apparatus preferably connects the aortic root with the heart apex and/or with the chest wall and/or with a rib after implantation. The shortening of the apparatus according to the present invention preferably leads to a pull on the aortic root and thus increases the displacement of the aortic root during the heart cycle. The increased displacement of the aortic root usually increases the restoring force of the aortic root, which in some cases leads to an increased retraction of the aortic root in the diastole and the associated increased pumping volume of the aortic root in the diastole.

In several embodiments, the apparatus according to the present invention is anchored to the fibrinous skeleton between the mitral valve ring and the tricuspid valve ring. The apparatus preferably increases the displacement of the heart skeleton and in particular of the entire heart base connected to it, synchronously with the heart cycle.

Preferably, the apparatus according to the present invention has no direct effect on myocardial contraction and on the portions of the heart which are remote from the center of the heart base and not connected to the fibrinous skeleton of the heart, such as the portions of the mitral and tricuspid valve annulus which are remote from the aortic root or the venous coronary sinus, in which no or only a rudimentary heart skeleton exists.

The aortic root is connected to the heart base and the fibrinous skeleton of the heart. The heart base is a structure in which the four heart valves lie in one plane. The heart base includes the heart skeleton, which is a structure of cartilaginous tissue that surrounds the aortic valve ring, the mitral valve ring, and the tricuspid valve ring.

When the heart contracts in the systole, the diameter of the ventricles decreases. In addition, the ventricles also shorten in their longitudinal direction. During the systole, the heart skeleton and the heart base with the four heart valves move synchronously with the aortic root towards the heart apex. In this, the aortic root is stretched and elastically dilated. During the diastole, the heart skeleton with the heart base is pulled by the elastic aortic root again in the opposite direction, away from the heart apex. During the entire heart cycle, however, the heart apex remains almost stationary, which is caused, among other things, by the pericardium. The pericardium is a closed sac that contains around 10 ml of fluid and surrounds the entire heart. The closed pericardium is fluid-tight and thus does, therefore, not allow fluid exchange with the surrounding body cavities. When the heart contracts, the pericardium, which is partially exposed freely in the chest, can follow the reduction in the circumference of the heart. However, the pericardium is connected to the manubrium sterni and the processus xiphoideus in the region of the heart apex by the sterno-pericardial ligament. During contraction of the heart, the pericardium and the heart apex lying in the closed pericardium cannot move from the chest wall towards the heart base. For this reason, the contraction of the heart muscle and the shortening of the heart lead to a pull on the base of the heart and the elastic aortic root, which is thus stretched in the systole. As soon as the heart muscle relaxes in the diastole, the stretched aortic root pulls the heart base away from the stationary heart apex due to the tension energy stored in it. Studies with sheep, which have anatomy comparable to humans with respect to the heart, show that, at rest due to tension, there is a force between the aortic root and the heart base of 1.8±0.2 N. To move the aortic root 10 mm towards the heart apex, a force F of 1.8±0.1 N must be applied. The normal deflection of the displacements(s) of the aortic root is 12±2 mm in healthy people. Thus, per heartbeat, to deflect the aortic root, work W of approximately

W=F*s=1.8N*0.012 m=0.0216 J

is required, wherein the corresponding energy is partly stored in the elastic stretch of the aortic root.

Thus, when the aortic root is stretched and lengthened in the systole, some of the energy of myocardial contraction is stored by the tension of elastic fibers. As the myocardium relaxes and after the aortic valve closes at the beginning of the diastole, the elastic fibers of the aortic root contract again and the aortic root moves away from the stationary heart apex and pumps blood into the body's circuit. This is an essential part of the windkessel function of the aortic root.

In an adult, about half of the stroke volume is temporarily stored in the aortic root, i.e., in the diastole, the aortic root pumps forward half the stroke volume with a time delay from the systole. In addition, the elastic retraction of the aortic root in the diastole causes suction in the left ventricle. With the retraction of the aortic root during the diastole, blood is sucked from the atria into the ventricles, which explains a brief negative pressure in both ventricles of up to −100 mmH2O (−7.3 mmHg) at the beginning of the diastole. This suction increases the blood volume in the ventricles at the end of the diastole or the preload and thus improves the ejection of the heart due to the Frank-Starling mechanism due to the shift of the Frank-Starling curve to the right. The retraction of the aortic root during the diastole also reduces the pressure in the aortic root at the beginning of the systole and thus reduces the afterload of the heart.

Heart weakness, in particular heart failure, occurs when the heart can only eject less than 45% of the ventricular volume. An ejection rate of less than 35% is considered a severe impairment.

Symptoms of heart failure include shortness of breath (exertional dyspnea, resting dyspnea, orthopnea, paroxysmal nocturnal dyspnea), fatigue, inadequate exhaustion after exertion, weakness, lethargy, fluid retention (leg or abdominal swelling, weight gain), nocturnal urination (nocturia), dry cough (especially at night), dizziness, syncope, loss of appetite, nausea, bloating, meteorism, constipation, abdominal pain, possibly weight loss, memory disorders, states of confusion and cognitive impairments.

Heart failure is classified according to severity into NYHA I (diagnosed heart disease without symptoms and without limitation of exercise capacity), NYHA II (mild limitation of exercise capacity, no symptoms at rest, but only with greater exercise), NYHA III (severe limitation of exercise capacity, no symptoms at rest, but already with mild exercise) and NYHA IV (persistent symptoms even at rest). When diagnosing heart failure, echocardiography is particularly important, with which regional and global limitations of heart function can be assessed.

Patients with heart failure with a left ventricular ejection fraction (EF) of less than 45% show a decreased displacement of the aortic root to less than 8 mm during the systole. The shortening of the long axis of the heart is a particularly sensitive parameter for measuring heart function. In these patients, during the systole, the aortic root is insufficiently moved towards the heart apex and insufficient tension energy is stored in the aortic root. Thus, no elastic restoring force is available during the diastole. As a result, the stroke volume is lost in the diastole, the afterload of the heart increases, and no suction is generated into the ventricles during the diastole.

In a heart failure with reduced ejection fraction (HFrEF), there is measured in echocardiography reduced wall motion of the heart with reduced ejection fraction of less than 45%. It can be seen that the shortening of both the long axis from the heart apex to the heart base and the short axis orthogonal to it is reduced. With two-dimensional echocardiography (Simpson method), the reduction in ejection volume can be determined.

At least half of the patients with symptoms of heart failure have normal cardiac function as measured by conventional echocardiography with a normal shortening of the orthogonal diameter of the ventricles. The ejection fraction calculated from this appears normal. Therefore, this form of heart failure is referred to as heart failure with preserved ejection fraction (HFpEF). Stiffening of the heart muscles and impaired diastolic function of the heart are discussed as causes for heart failure. However, a closer echocardiographic examination of the cardiac function of these patients often reveals a change in the twisting and untwisting of the left ventricle as well as a reduction in the diastolic suction and the early diastolic filling of the left ventricle.

The normal movement of the aortic root with the heart base towards the heart apex of 12±2 mm is reduced to significantly less than 8 mm in HFpEF. The speed of the stroke of the aortic root in the systole and the early diastole is highly significantly reduced with 0.64±0.51 cm/s compared to 1.54±0.51 cm/s in the systole and 1.49±0.77 cm/s compared to 2.32±1.24 cm/s at the start of the diastole. The poor performance and the symptoms of heart failure in these patients correlate with a lower level of elasticity, in particular a stiffening of the aortic root. At the same time, thickening of the myocardium is found in these patients, suggesting compensation for the lower windkessel function and a smaller stroke of the aortic root. The heart force still appears normal or even increased in echocardiography, but is no longer sufficient to pull the aortic root towards the heart apex, since this is less elastic and an increased force would be required to bring about a sufficient stroke of the aortic root.

The apparatus according to the present invention should preferably be used in chronic heart failure with preserved left ventricular ejection fraction (HFpEF), in which the systolic heart muscle function is largely preserved, but the cardiac strength is no longer sufficient to stretch an aortic root stiffened by various diseases or aging processes and, associated therewith to produce a sufficient stroke of the heart base or stretching of the aortic root.

In several embodiments according to the present invention, the apparatus is provided for supporting the heart action by displacing the heart base and/or the aortic root and comprises at least one lifting drive.

In several embodiments according to the present invention, the apparatus is designed for the at least partial implantation in a heart.

In several embodiments according to the present invention, the apparatus comprises a first anchor for implantation in or on the heart base, the heart skeleton, the aortic root and/or a structure in local proximity to the aortic root.

In several embodiments according to the present invention, the apparatus comprises a second anchor for implantation in or on the heart apex, a ligament, a rib, a sternum, and/or a structure in local proximity to the heart apex.

In several embodiments according to the present invention, the first anchor for implantation in or on the heart base comprises a bracket which can be implanted such that it extends from the left atrium into the right atrium and penetrates the lower end of the interatrial septum between the mitral valve annulus and tricuspid valve annulus.

In several embodiments according to the present invention, the apparatus is designed to shorten during the systole and to lengthen during the diastole.

In several embodiments according to the present invention, the first anchor is an implant, in particular an intravascular stent, in the aorta or an aortic valve prosthesis.

In several embodiments according to the present invention, the energy that the apparatus uses to support the heart action is tension energy of an elastic element of the apparatus.

In several embodiments according to the present invention, the force that is supplied to the apparatus for supporting the heart action is transferred mechanically or magnetically from an energy source via a connection unit.

In several embodiments according to the present invention, the force used by the apparatus to support the heart action is a pneumatic or hydraulic force.

In several embodiments according to the present invention, the apparatus comprises an energy source which provides the energy required to support the heart action as electrical, pneumatic, hydraulic or magnetic energy.

In some embodiments of the present invention, the apparatus is bistable between an upward diastolic position and a downward systolic position.

In several embodiments according to the present invention, the apparatus comprises a control unit.

In several embodiments according to the present invention, the insertion system comprises an insertion catheter, a guiding catheter, a guiding wire, and/or at least one delivery or supply catheter.

In several embodiments according to the present invention, a method for controlling an apparatus according to the present invention comprises the steps of providing an apparatus according to the invention with a control unit and controlling the apparatus according to the present invention using the control unit in order to support the heart action.

In several embodiments according to the present invention, a digital storage medium with electronically readable control signals is configured in order to interact with a programmable computing unit such that the machine steps of the method for controlling an apparatus according to the present invention are prompted.

In several embodiments according to the present invention, a computer program product, as a signal wave or with a program code stored on a machine-readable carrier, is provided to prompt the machine steps of the method for controlling an apparatus according to the present invention when the computer program product runs on a computing unit.

In several embodiments according to the present invention, a computer program with a program code for initiating the machine steps of the method for controlling an apparatus according to the present invention is provided when the computer program runs on a computing unit.

In several embodiments according to the present invention, the connecting piece (also: anchor) spans a first main extension plane alone or together with one or more guiding devices. In certain embodiments, the apparatus does not have a structure, in particular it has no extension or extension unit, which lies in a second main extension plane intersecting the first main extension plane at an angle between 70° and 110°.

In some embodiments according to the present invention, the guiding device(s) of the apparatus is/are not provided in order to be guided, positioned in or fixed in the (already existing) valve openings of the heart, in particular through the mitral valve opening and/or the tricuspid valve opening.

In several embodiments according to the present invention, the mitral valve is not penetrated by one of the guiding devices behind the posterior mitral valve leaflet.

Several of the embodiments of the present invention may have the following advantages:

Preferably, the apparatus according to the present invention supports the stretch and stroke of the aortic root.

With a larger stroke of the aortic root, the elastic pre-stretching of the aortic root is increased in the systole and a larger elastic retraction of the aortic root occurs during the diastole. This increases the amount of blood that is carried forward during the diastole by the elastic retraction of the aortic root (windkessel function).

With a larger stroke of the aortic root, the retraction of the aortic root is enhanced during the diastole and the suction in both ventricles is increased during the diastole, resulting in better emptying of the atria.

With a larger stroke of the aortic root, the long axis of the left and right ventricles is shortened more during the systole, and more blood can be ejected from both ventricles.

The increase in the stroke of the aortic root and the associated heart skeleton or heart base supports the passive pumping capacity of the aortic root and the pulmonary root in the diastole as well as the active pumping capacity of the right and left ventricle in the systole. By increasing the end-diastolic suction in the ventricles, the preload of both ventricles is increased and the Frank-Starling curve is shifted to the right. This lowers the end-diastolic pressure in the atria. By enhancing the windkessel function in both the aortic root and the pulmonary root, the afterload of both ventricles is decreased, thus increasing the ejection volume of both ventricles.

A support of the aortic root stroke may have a positive effect on the diastolic function of the heart. A reduced elasticity and restoring force of the aorta reduces the diastolic suction, the end-diastolic filling and thus the preload and, as a result, the performance of the left ventricle according to the Frank-Starling mechanism.

Supporting the stretching of the aorta during the systole allows a higher storage of tension energy in the aorta, which leads to an increased retraction of the aortic root in the diastole and thus an increased diastolic pull in the left ventricle. This results in improved emptying of the atria and in increased filling and preload of the ventricles, which according to the Frank-Starling mechanism improves heart output.

The aortic root stroke has a positive effect on the Windkessel function of the aortic root and aorta. The aortic root is a cylinder with an average diameter of 3.5 cm. In a healthy person, this cylinder is stretched by a length of approximately 12 mm during the systole. The volume of the cylinder is V=πr²h=11.55 ml. This volume is pumped forward during diastole. This corresponds to 16.5% of an average stroke volume of 70 ml. This does not yet take into account the radial stretching and windkessel function of the aorta, which are also reduced with a reduced elasticity of the aortic root. Supporting the stretching aortic stroke leads to at least partial restoration of the windkessel function in relation to the shortening of the aorta in the diastole.

In patients with HFpEF, the stretching and stroke of the stiffened aortic root are reduced. In these patients, heart strength is normal or compensatory enlarged. The lack of shortening of the heart chambers during the systole cannot be compensated for by an additional reduction in the diameter of the ventricle and causes a reduction in the ejection volume. Supporting the aortic root stroke by the apparatus according to the present invention may advantageously lead to a shortening of the longitudinal axis of the heart and thus to an improved ejection of the heart during the systole. A positive effect of the apparatus according to the present invention on the muscle contraction of both ventricles and both atria may be a side effect of the increased aortic root stroke.

By supporting the longitudinal stretching of the aortic root, the stroke volume of the aortic root cylinder described above is preferably increased and the blood volume pumped forward in the diastole is increased. As a result, the pressure in the aortic root at the beginning of the systole is correspondingly reduced and the afterload of the heart is decreased, thereby increasing the stroke volume.

In several embodiments, the apparatus according to the present invention, in particular the first anchor of the apparatus, is not embodied to be attached to the mitral valve (in particular its ring) or to be attached exclusively to the mitral valve (in particular its ring). In several embodiments, the device is not designed to be attached to a mitral valve prosthesis.

In several embodiments, the apparatus does not include a mitral valve prosthesis.

In some embodiments, the apparatus according to the invention, in particular the first anchor of the apparatus, does not have a closed circular or annular shape, nor does it have the shape of a partial circle (or partial annulus) whose circular angle exceeds 180°. It is not shaped as a bow-tie or as a loop, in particular it does not have the form of a loop-shaped annuloplasty, not even a partial annuloplasty (of a mitral valve).

In several embodiments, the apparatus according to the present invention does not have the shape of a circle or of a partial circle (or of a ring or of a partial ring) the main extension plane of which one or more structures, e.g. extensions or extension units, e.g. spoke-shaped, are arranged.

In some embodiments, no guiding device begins or ends in the inner region of the ring or partial ring (or in a region enclosed by it). Also there, in some embodiments, there is no guiding device connected, or provided to be connected, to an end section of the connecting piece.

In several embodiments, the apparatus does not have a portion intended to rest on the edge of a heart valve, e.g., mitral valve or mitral valve prosthesis.

In some embodiments, the apparatus does not have a structure that is intended to remain in the heart while extending through the flow-through cross-section of a heart valve.

In several embodiments, the apparatus according to the present invention is designed to have more than 50%, preferably more than 75%, more preferably more than 85% of its length within the (left and/or right) ventricle and/or the (left and/or right) atrium.

In several embodiments, the apparatus according to the present invention is designed to be anchored in the heart wall exclusively at one side of the heart, in particular exclusively at the heart apex, in particular to penetrate the heart wall.

In several embodiments, the apparatus is not designed to be simultaneously anchored to opposing portions of the heart wall.

In several embodiments, the apparatus is designed to be arranged, after insertion, with at least 30%, preferably at least 50%, particularly preferably at least 70% of its mass and/or its volume outside the left heart, in particular outside the left ventricle.

In several embodiments, the apparatus does not have a permanent magnet and/or an electromagnet.

In the following, the apparatus according to the invention is described on the basis of preferred embodiments thereof with reference to the attached drawings. However, the invention is not limited to these embodiments. In the drawings:

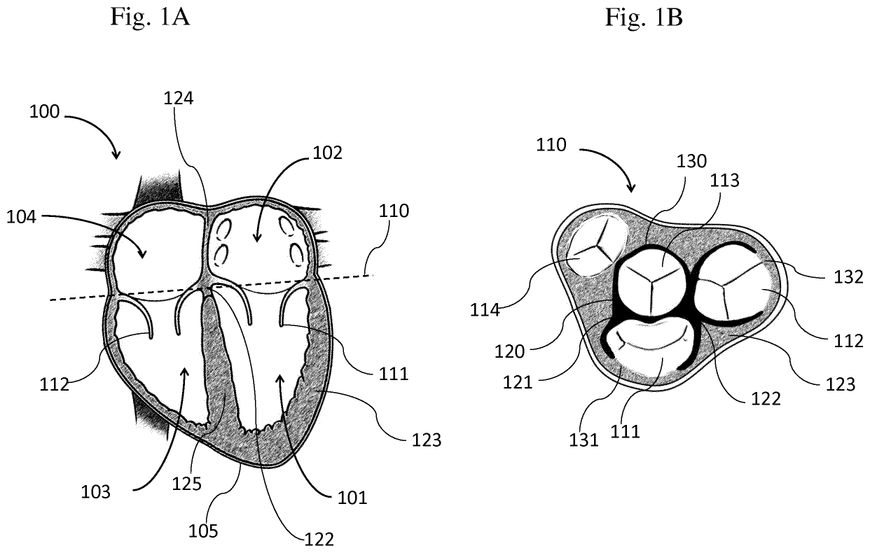

FIG. 1 shows a representation of a heart with the four ventricles, heart base and heart skeleton;

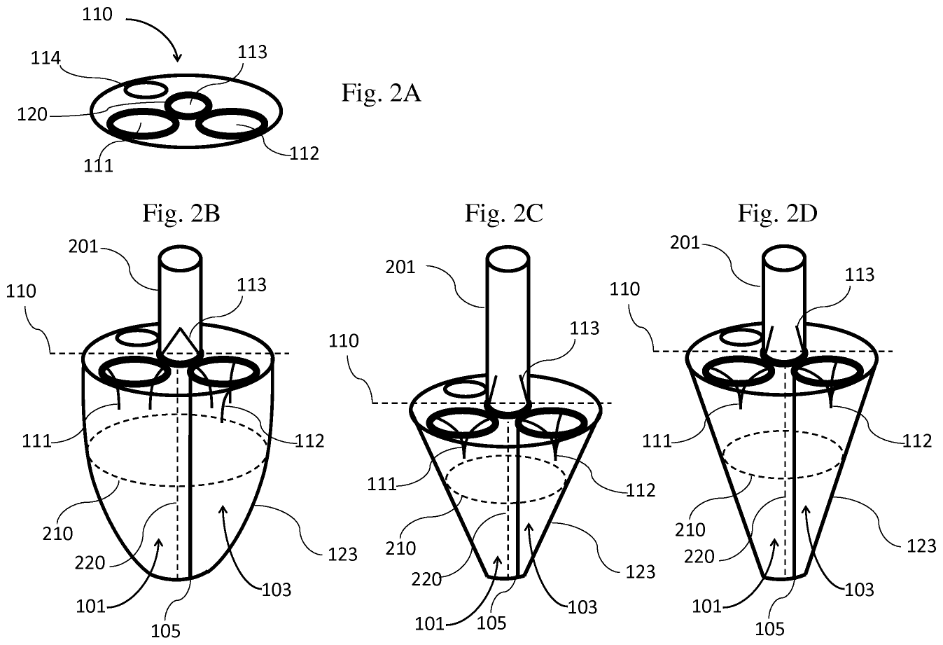

FIG. 2 shows a representation of the pumping function of the heart;

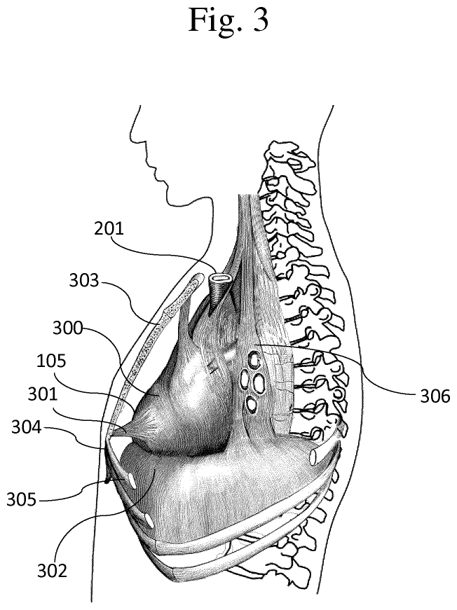

FIG. 3 shows a representation of the suspension of the heart sac (or pericardium) in the chest (or thorax);

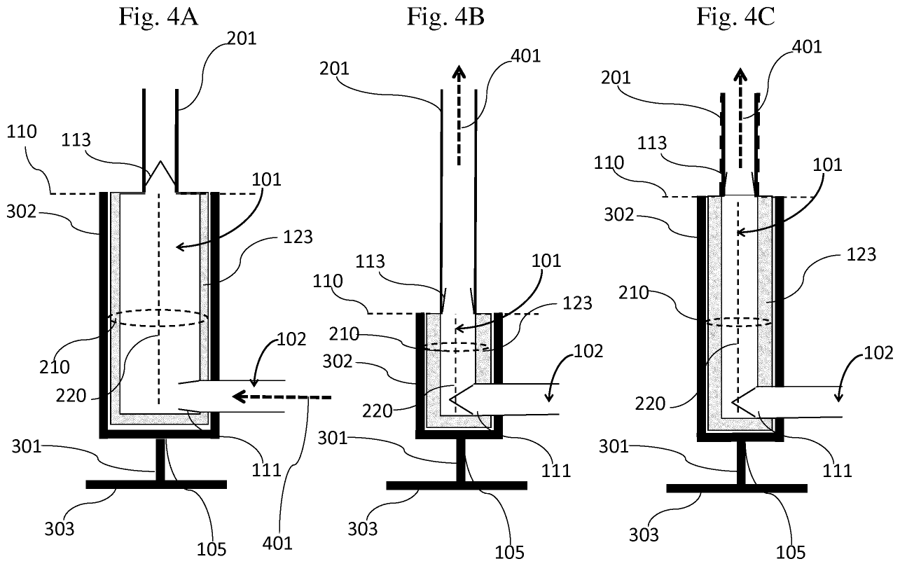

FIG. 4 shows a schematic representation of the pumping function of the left ventricle;

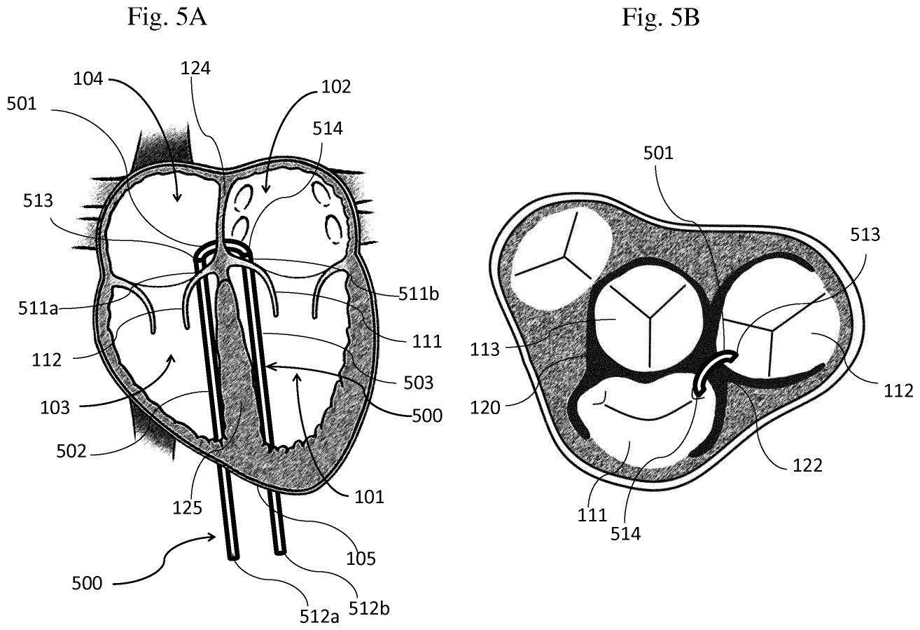

FIG. 5 shows an embodiment of the apparatus according to the present invention for supporting the aortic stroke;

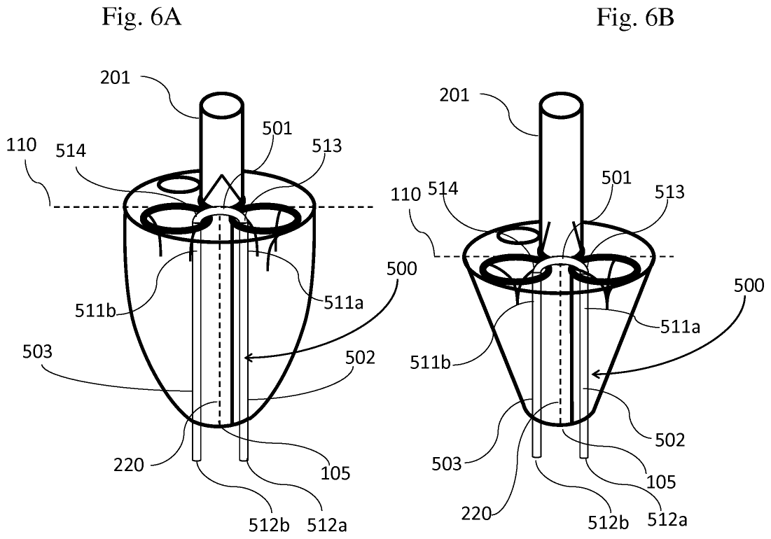

FIG. 6 shows a schematic representation of the function of an embodiment of the apparatus according to the present invention for supporting the aortic stroke;

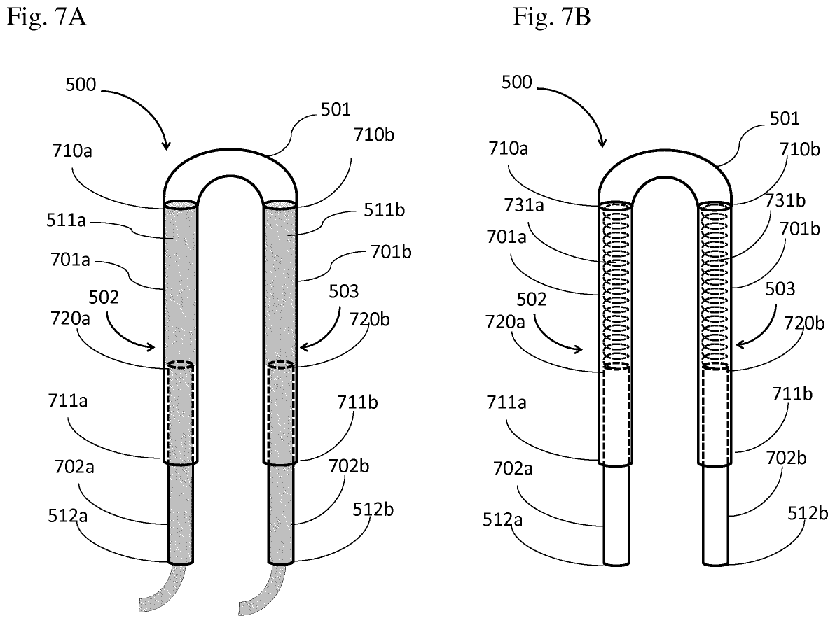

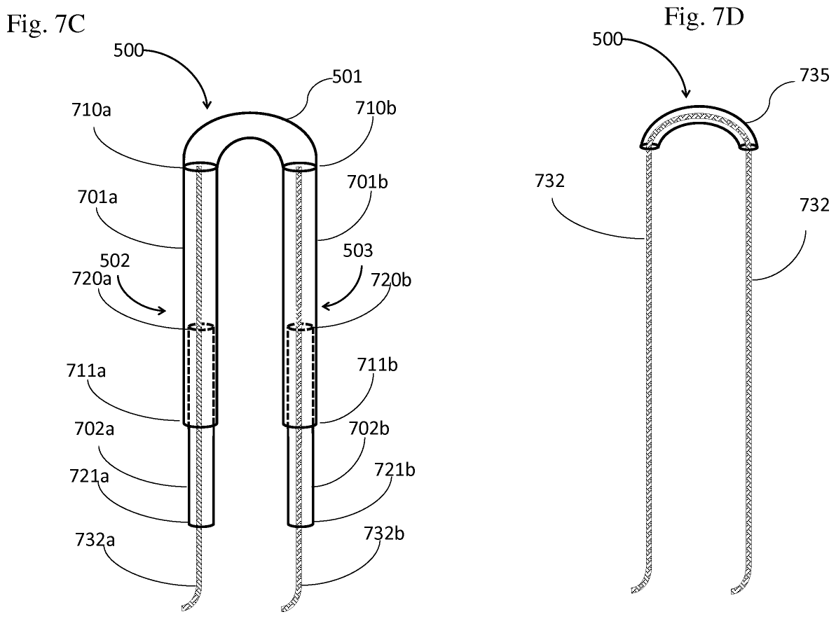

FIG. 7 shows the apparatus according to the present invention in an embodiment;

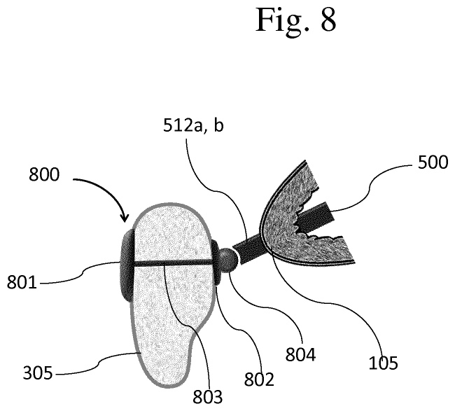

FIG. 8 shows a second anchor of an embodiment of the apparatus according to the present invention which is connected to a rib; and

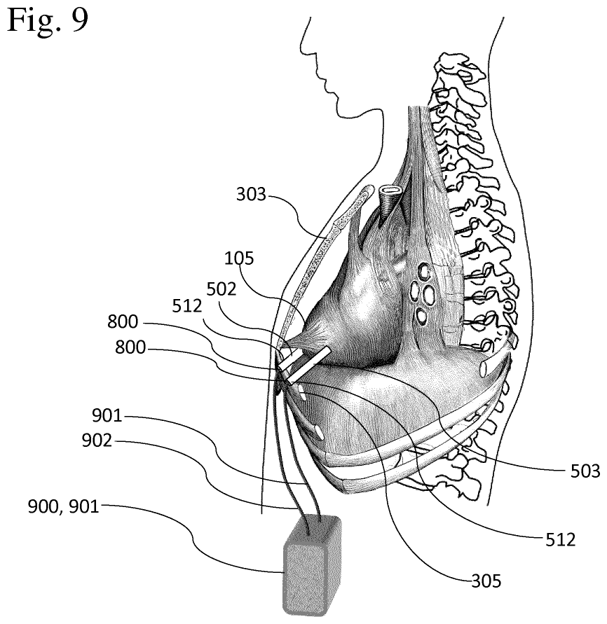

FIG. 9 shows the apparatus according to the present invention in the implanted state in the thorax with a control device according to the present invention.

FIG. 1 shows a sectional view of a human heart 100 with the four ventricles (ventricle or atrium) 101, 102, 103, 104 and the heart base 110. FIG. 1A shows the left ventricle 101 (left lower heart chamber 101) and the left atrium 102 (left upper heart chamber 101) with the intermediate mitral valve 111 disposed in between, the right ventricle 103 (right lower heart chamber 103) and the right atrium 104 (right upper heart chamber 104) with the intermediate tricuspid valve 112 disposed in between. The interatrial septum 124 is disposed between the two atria 102, 104, and the interventricular septum 125 is disposed between the two ventricles 101 and 103.

In FIG. 1B, the heart base 110 is shown. The heart base 110 is a comparatively flat anatomical structure of the heart 100, on which the two atrioventricular valves, namely the mitral valve 111 and tricuspid valve 112, and the two pocket valves, namely the aortic valve 113 and the pulmonary valve 114, are disposed. The aortic valve 113 is enclosed by the aortic valve ring 130 or fixed in the aortic valve ring 130. The heart skeleton 120 consists of cartilaginous tissue and is the only rigid structure of the heart 100. The heart skeleton 120 completely encompasses the aortic root 201 and the central parts of the mitral valve ring 131 and the tricuspid valve ring 132. The most vigorously formed portions of the heart skeleton 120 are the left fibrous trigone 121 and the right fibrous trigone 122. On the right fibrous trigone 122, the mitral valve 111 and the tricuspid valve 112 adjoin each other. The heart muscle 123 of the interventricular septum 125 is connected to the heart skeleton 120 in the area of the right fibrous trigon 122 between the mitral valve 111 and the tricuspid valve 112. At this point, the contraction of the heart muscle 123 of the interventricular septum 125 leads to a pull or traction on the heart skeleton 120 and the therewith associated aortic valve 113 towards the heart apex.

The mitral valve 111 and tricuspid valve 112 close at the end of the diastole and ensure that when the ventricles 101 and 103 contract, the blood does not flow back into the atria 102 and 104 but is pumped forward and, on the right side, into the lungs and, on the left side, into the body's circuit. The aortic valve 113 and the pulmonary valve 114 close at the end of systole and ensure that, after the contraction of the two ventricles 101 and 103, the blood does not flow back into the ventricles 101 and 103, but rather that the diastolic blood pressure is maintained in the pulmonary artery and lungs on the right side and that the diastolic pressure is maintained in the aorta and the body's circuit on the left side.

FIG. 2 shows a schematic representation of the heart. FIG. 2A shows the heart base 110 schematically. FIG. 2B shows the heart 100 in the diastole with relaxed heart muscle 123. The longitudinal axis 220 is longest here in the contraction cycle and the circumference of the ventricles 210 and the orthogonal diameter are largest. The two ventricles 101 and 103 are filled; the mitral valve 111 and tricuspid valve 112 are open to allow blood inflow into the ventricles. The aortic valve 113 is closed and thus prevents the backflow of blood from the body's circuit into the left ventricle 101 and maintains the diastolic blood pressure in the body. The aortic root 201 is maximally contracted or shortened at the end of the diastole.

FIG. 2C schematically shows the contraction of heart 100 in the systole with a normally elastic aortic root 201. The heart muscle 123 is contracted, the heart 100 has the smallest circumference 210 and the smallest orthogonal diameter in the heart cycle. The heart apex 105 remains stationary, the heart base 110 has shifted to the heart apex 105, and the longitudinal axis 220 is the shortest. The atrioventricular valves 111 and 112 are closed in order to prevent a back flow of blood into the two atria 102 and 104. The aortic valve 113 is open to allow ejection of blood into the body's circuit. The aortic root 201 is maximally stretched.

FIG. 2D schematically shows the contraction of the heart 100 in the systole as well, but with a stiff, non-elastic aortic root 201. The heart muscle 123 is contracted, and the heart has the smallest circumference 210 and the smallest orthogonal diameter with regard to the heart cycle. The heart apex 105 remains stationary, the position of the heart base 110 has not changed in relation to the diastole, since the stiff aortic root 201 cannot be stretched and so the heart base 110 cannot be drawn towards the heart apex 105. The longitudinal axis 220 is as long as in the relaxed heart 100 in diastole in FIG. 2B. However, due to muscle contraction, the circumference 210 and the orthogonal diameter are the smallest with regard to the heart cycle. The atrioventricular valves 111 and 112 are closed to prevent a backflow of blood into the two atria 102 and 104. The aortic valve 113 is open to allow ejection of blood into the body's circuit.

FIG. 3 shows a schematic representation of the chest of a person. The pericardium 300 (heart sac) rests on the diaphragm 302 and is stretched between the aortic root 201 with the mediastinum 306 and the sterno-pericardial ligament 301. The sterno-pericardial ligament 301 extends from the pericardium 300 in the area of the heart apex 105 to a rib 305 and to the manubrium sterni at the end of the sternum 303.

As the heart contracts, the pericardium 300 may follow the reduction in heart circumference 210 during the systole. Since the closed pericardium is stretched between the relatively immobile sternum 303 and the mediastinum 306 via the sterno-pericardial ligament 301, the heart apex 105 cannot move away from the sternum 303 in the direction of the heart base 110. For this reason, contraction of the heart and shortening of the longitudinal axis 220 of the heart results in traction on the heart base 110 and the elastic aortic root 201, which is thus stretched in the systole and the heart base 101 with the aortic root 201 is pulled toward the heart apex 105.

FIG. 4 shows a schematic representation of the pumping function of a ventricle 101 as depending on the elasticity of the aortic root 201.

FIG. 4A schematically shows a left ventricle 101 in diastole. The heart muscle 123 is relaxed, the longitudinal axis 220 is the longest, the circumference of the ventricle 210 and the orthogonal diameter are the largest in the heart cycle. The ventricle 101 is filled and the mitral valve 111 is open to allow the inflow of blood 401 into the ventricle. The aortic valve 113 is closed and thus prevents the backflow of blood from the body's circuit into the left ventricle and maintains the diastolic blood pressure in the body. The aortic root 201 is maximally contracted at the end of the diastole.

FIG. 4B schematically shows the contraction of the heart with a normally elastic aortic root 201. The heart muscle 123 is contracted, the heart has the smallest circumference 210 or orthogonal diameter. The heart apex 105 remains stationary since the pericardium 300 is connected to the sternum 303 via the sterno-pericardial ligament 301. Due to the contraction of the heart muscle 123, the heart base 110 has shifted to the heart apex 105 and the longitudinal axis 220 is the shortest in the heart cycle. The mitral valve 111 is closed to prevent the backflow of blood into the atrium 102. The aortic valve 113 is open to allow ejection of blood 401 into the body's circuit. The aortic root 201 is maximally stretched. After the systole in FIG. 4B, the heart muscle 123 relaxes, the circumference of the heart 210 and its orthogonal diameter increases again, and the aortic root 201 pulls the heart base 110 away from the heart apex 105 (FIG. 4A). By this, blood present in the aortic root 201 is pumped into the body's circuit during the diastole.

FIG. 4C schematically shows contraction of the left ventricle 101 as well, but with a stiff, nonelastic aortic root 201. The heart muscle 123 is contracted. The heart apex 105 remains stationary since the pericardium 300 is connected to the sternum 303 via the sterno-pericardial ligament 301. The position of the heart base 110 has not changed since the stiff aortic root 201 cannot be stretched and thus the heart base cannot be drawn towards heart apex 105. The longitudinal axis 220 is as long as that in the relaxed heart in diastole in FIG. 4A. However, because of the muscle contraction, the circumference 210 and the orthogonal diameter are smallest in the heart cycle. The mitral valve 111 is closed in order to prevent backflow of blood into the left atrium 102. The aortic valve 113 is open to allow ejection of blood into the body's circuit.

After the systole FIG. 4C, the heart muscle relaxes again and the circumference 210 increases again (FIG. 4A). Since the heart base 110 remains stationary, there is no forward pumping of blood in the aortic root 201 with stiff aortic root 201 in FIG. 4C during the diastole compared to the situation with elastic aortic root in FIG. 4B.

FIG. 5 shows an exemplary embodiment of the apparatus 500 according to the present invention. In this, FIG. 5A shows a schematic representation of the apparatus 500 in the ventricles 101 and 103 and in the atria 102 and 104. The apparatus 500 comprises a first anchor 501, which can be referred to as bracket or connecting piece, and a guiding device 502, which can be referred to as a lifting drive, which is optionally arranged in the right ventricle 103, and a guiding device 503, which is optionally arranged in the left ventricle 101.

In several embodiments, the length of the apparatus 500 according to the present invention is preferably between 70 mm and 120 mm in the diastole, particularly preferably between 72 mm and 116 mm, and preferably between 60 mm and 115 mm in the systole, particularly preferably between 62 mm and 114 mm. The change in length of the apparatus 500 from diastole to systole is preferably a minimum of 0 mm and a maximum of 21 mm, particularly preferably a minimum of 10 mm and a maximum of 15 mm.

The first anchor 501 comprises two ends 513 and 514. A first end 513 is optionally arranged in the implanted state at the lower end of the right atrium 104 in the immediate vicinity of the base of the septal leaflet of the tricuspid valve 112. A second end 514 of the first anchor 501 is optionally arranged in the implanted state at the lower end of the left atrium 102 in the immediate vicinity of the base of the anterior leaflet of the mitral valve 111. The first anchor 501 optionally penetrates the atrial septum 124 at the lower end of the atrial septum 124 and, as shown in FIG. 5B, above the right fibrous trigone 122 where the mitral valve 111 and the tricuspid valve 112 adjoin each other.

The first anchor 501 is connected at its first end 513 (When referring to an end herein, this may optionally be understood as an end region. These two terms may optionally be interchanged.) in the right atrium 104 to the first end 511 of the guiding device 502. The first end 511 of the guiding device 502 penetrates the septal leaflet of the tricuspid valve 112 directly at the base of the valve leaflet. The guiding device 502 is located in the right ventricle 103, directly adjacent to the interventricular septum 125 and penetrates the heart apex 105 with its second end 512a. The second end 512a of the guiding device 502 is optionally connected to a rib 305 (see FIGS. 8 and 9); alternatively, the second end 512a may be anchored, for example, directly in heart apex 105 or on the sterno-pericardial ligament 301.

The first anchor 501 is connected at its second end 514 in the left atrium 102 to the first end 511b of the second guiding device 503. For simplicity, the second guiding device 503 will also be referred to as guiding device hereinafter. The first end 511b of the guiding device 503 has penetrated the anterior leaflet of the mitral valve 111 directly at the base of the valve leaflet. The second guiding device 503 is located in the left ventricle 101, directly next to the interventricular septum 125 and penetrates with its second end 512b into the heart apex 105, preferably all the way through it. The second end 512b of the second guiding device 503 is optionally connected to a rib 305; alternatively, the second end 512b can be anchored directly in the heart apex 105 or on the sterno-pericardial ligament 301.

In several embodiments, the apparatus 500 according to the present invention may be implanted entirely surgically via open heart surgery. Alternatively, in several embodiments, the apparatus 500 according to the present invention may be implanted via a combination procedure using a catheter and surgical intervention. Alternatively, in several embodiments, the apparatus 500 according to the present invention may also be introduced completely by a catheter.

The complete surgical implantation is preferably carried out via a conventional heart operation.

Alternatively, in several embodiments, the implantation may be performed by performing or carrying out a combination procedure in which a catheter is used to introduce the first anchor 501 into the atrial septum via the vena cava and the right atrium 104. The chest is surgically opened over the heart apex 105, and the first two ends 511a and 511b of the guiding devices 502 and 503 are inserted over the apex 105 into the beating heart along the interventricular septum 125. In this case, the guiding devices 502 and 503 perforate the tricuspid valve 112 and mitral valve 111 from the ventricular side. The two first ends 511a, 511b of the guiding devices 502, 503 are then connected to the corresponding end 513 or 514, respectively, of the first anchor 501 under fluoroscopy at the beating heart. The second ends 512a, 512b of the guide devices 502, 503 are then openly surgically connected to a rib. Alternatively, the second ends 512a, 512b may be anchored directly in the heart apex 105 or the sterno-pericardial ligament 301.

In the fully catheter-based implantation, the guiding device 502 is inserted into the heart via the right atrium 104 and the septal leaflet of the tricuspid valve 112 is perforated with the second end 512a of the guiding device 502. The guiding device 502 is advanced into the heart apex 105 and is anchored there. Alternatively, the heart apex 105 can be perforated with the second end 512a of the guiding device 502 and then anchored in the sterno-pericardial ligament 301 or on the rib 305.

The second guiding device 503 is introduced into the heart via the right atrium 104 and advanced into the left atrium 102 via a perforation in the atrial septum 124. The anterior mitral valve leaflet 111 is perforated with the second end 512b of the guiding device 503. The second end 512b of the guiding device 503 is advanced into the heart apex 105 and anchored there. Alternatively, the heart apex 105 may be perforated with the second end 512a of the guiding device 502 and then anchored in the sterno-pericardial ligament 301 or on the rib 305.

The first anchor 501 is inserted into the heart via the right atrium 104, and the second end 514 is inserted into the left atrium 102 via a perforation in the interatrial septum 124. Under fluoroscopy, a closure system (not shown) is connected between the first end 514 of the connector 501 to the first end 511b of the guiding device 503 and between the second end 513 of the first anchor 501 to the first end 511a of the guiding device 502.

FIG. 6 schematically represents the function of the apparatus 500 according to the present invention, after it has been implanted in a heart, with a stiff aortic root 201 as has already been described in FIG. 4C. It is assumed that the heart's strength is insufficient to stretch the stiff aorta 201 and pull the heart base 110 toward the heart apex 105. In FIG. 4B, a normally elastic aorta 201 was described in which the heart base 110 may be pulled toward the heart apex 105.

FIG. 6A shows schematically the heart in the diastole with the implanted apparatus 500 according to the present invention.

The guiding devices 502 and 503 are stretched, the long axis of the heart 220 is longest in the heart cycle, the heart base 110 is farthest from the heart apex 105, and the aortic root 201 is correspondingly short.

FIG. 6B shows the heart at the end of the systole schematically. It is assumed that the heart's strength is insufficient to stretch the stiff aortic root 201. The guiding devices 502 and 503 have actively shortened and pulled the heart base 110 and thus also the stiff aortic root 201 to the heart apex 105. As a result, the longitudinal axis 220 is shortest in the heart cycle and the stiff aortic root 201 is actively stretched by the apparatus 500 according to the present invention.

On the way back from systole in FIG. 6B to the diastole in FIG. 6A, the aortic root 201 contracts again. As a result, the heart base 110 moves away from the heart apex 105 and the corresponding column of blood in the aortic root 201 is pumped into the body's circuit during the diastole.

FIG. 7 shows, schematically simplified, alternative embodiments of the apparatus 500 according to the present invention. In FIG. 7A, the guiding devices 502 and 503 are shortened by hydraulic force. The shortening occurs by moving the linear guiding devices 702a, 702b, optionally designed as pistons, relative to the linear guiding sleeves 701a, 701b, designed as cylinders. If it is shortened, the pistons are pushed into the cylinder; if it is lengthened, the pistons are pulled out of the cylinders. The second ends 512a, 512b of the pistons 702a, 702b may be anchored for example to the heart apex 105, to the sterno-pericardial ligament 301 or to the rib 305. By applying a negative pressure to the respective second end 512a, 512b of the pistons 702a, 702b, the first ends 710a, 710b of the cylinders 701a, 701b move towards the first ends 720a, 720b of the pistons 702a, 702b. This results in a shortening of the guiding devices 502 and 503 and the first anchor 501 is pulled towards the heart apex 105.

In FIG. 7B, the guiding devices 502 and 503 are shortened by a tension caused by a spring 731a, 731b. The second end of the piston 512 is anchored to the heart apex 105, to the sterno-pericardial ligament 301, or to the rib 305. In each of the guide devices 502 and 503 there is a spring 731a, 731b which connects the first end of the cylinder 710a, 710b to the first end 720a, 720b of the piston 702a, 702b. As a result of the retraction of the aortic root 201 in the diastole, the pistons 702a, 702b in the cylinders 701a, 701b are moved to the second end of the cylinder 711a, 711b and the springs 731a, 731b are tensioned. The springs 731a, 731b may be referred to as tension springs. During the systole, the springs 731a, 731b shorten and the first ends 710a, 710b of the cylinders 701a, 701b are drawn towards the first end of the pistons 720a, 720b. In this way, the increased force for stretching the stiff aorta 201 in the systole is at least partially compensated for by the tension of the springs 731a, 731b.

In FIG. 7C, the guiding devices 502 and 503 become shorter with the aid of a rope 732. The second ends 512a, 512b of the pistons 702a, 702b may be anchored to the heart apex 105, to the sterno-pericardial ligament 301, or to the rib 305. In the cylinders 701a, 701b, the rope 732a, 732b is attached to the first end of the cylinder 710a, 710b, respectively. The end of the rope 732a, 732b is redirected at a second anchor (not shown) in the vicinity of the heart apex 105, of the sterno-pericardial ligament 301 or of the rib 305.

By pulling on the ropes 732a, 732b, the cylinders 701a, 701b are pulled over the respective pistons 702a, 702b and the respective first end 710a, 710b of the cylinders 701a, 701b moves to the first ends 720a, 720b of the pistons 702a, 702b. This results in a shortening of the guiding devices 502 and 503 and the connecting piece 501 is pulled towards the heart apex 105.

In FIG. 7D, an apparatus 500 according to the present invention is shown, which consists only of a first anchor 501 designed as a hollow connecting piece 735 and a rope 732. The end of the rope 732 is redirected at a second anchor (not shown) at the heart apex 105, at the sterno-pericardial ligament 301, or at the rib 305. Pulling on one or both ends of the rope 732, results in the pulling of the connector 735 toward the heart apex 105.

In addition to the apparatuses 500 described, other active shortening mechanisms are also conceivable and encompassed by the invention, such as shortening by magnetic force, by a transplanted muscle, a bioengineered muscle, or an artificial muscle, each of which may be used to generate force.

FIG. 8 represents a second anchor 800 on the rib 305. The anchor 800 is attached with an abutment 801 on the outside of the rib 305, an abutment 802 on the inside of the rib 305, and with a central connection 803 through the rib 305. The second anchor 800 is secured in local proximity to the heart apex 105 in the rib 305. A ball joint 804 movably connects the second anchor 800 to the second end 512a, 512b of the guiding device 502, 503. Preferably, both guiding devices 502 and 503 are movably connected to the rib 305 with their second end 512a, 512b, by a second anchor 800, respectively.

In addition to the described second anchor 800 on the rib, other designs and anchor shapes are conceivable (e.g. also merely the use of a surgical thread), which are also encompassed by the present invention.

FIG. 9 represents a chest with an implanted apparatus 500 according to the invention. The two second ends 512a, 512b of the guiding devices 502 and 503 are each connected to the rib 305 by a second anchor 800. The respective guiding device 502 and 503 is connected to the control unit 901 and/or to the energy source 900 via the connections 902. Depending on the design, the required lifting force of the guiding device is transferred via the connections 902 as hydraulic force, tensile force, rotational force, electromagnetic force or in another form. The control unit 901 may be synchronized with natural heart activity via an ECG measurement or a pressure measurement. The control unit 901 may include an electric motor, linear motor, gears, pumps, or other devices necessary to generate and transmit the required force through the connections 902. The control unit 901 may include power sources such as batteries or rechargeable batteries that can be charged via an external charger. Other energy sources, such as nuclear energy, may also be conceivable to provide the necessary power and are encompassed by the present invention.

### LIST OF REFERENCE NUMERALS

100 heart
101 left ventricle,
102 left atrium
103 right ventricle,
104 right atrium
105 heart apex
110 heart base
111 mitral valve
112 tricuspid valve
113 Aortic valve
114 pulmonary valve
120 heart skeleton
121 left fibrous trigone
122 right fibrous trigone
123 heart muscle
124 interatrial septum
125 interventricular septum, ventricle septum
130 aortic valve ring
131 mitral valve ring
132 tricuspid valve ring
201 aortic root
210 circumference of the ventricles in the diastole, orthogonal diameter
220 longitudinal axis of the ventricles
300 pericardium, heart sac
301 sterno-pericardial ligament
302 diaphragm, midriff
303 sternum
304 xiphoid process
305 rib
306 mediastinum, mediastinal area
401 blood flow or influx
500 apparatus for supporting heart action
501 first anchor; bracket; connecting piece between the two guiding devices
502 guiding device; lifting drive
503 guiding device; second guiding device;
511a,b first end of the guiding device
512a,b second end of the guiding device
513 first end of connecting piece 501 in the right atrium
514 second end of the connecting piece 501 in the lift atrium
701a,b cylinder; linear guiding sleeve
702a,b piston; linear guiding device
710a,b first end of the cylinder
711a,b second end of the cylinder
720a,b first end of the piston
721a,b second end of the piston
731a,b spring
732 pulling device; rope
732a,b pulling device; rope
735 first anchor; connecting piece between the atria
800 second anchor; optionally arranged on the rib
801 abutment on the outer side of the rib
802 abutment on the inner side of the rib
803 connection of the abutment on the outer side of the rib with the abutment on the inner side of the rib
804 ball joint
900 energy source
901 control unit
902 connection of control unit with guiding device

1. An apparatus for supporting the heart action, comprising at least a first anchor and a pulling device or guiding device for displacing the first anchor, wherein the first anchor is provided and configured for implantation in or on the heart base, the heart skeleton, the aortic root and/or a structure in local proximity to the aortic root, and/or said apparatus encompassing at least one lifting drive.

2. The apparatus according to claim 1, wherein the pulling device or guiding device is, or comprises a linear guiding device.

3. The apparatus according to claim 2, wherein the first anchor comprises a V- or U-shaped section and the first anchor is connected, with or by at least one linear guide sleeve, to at least one end of the first anchor, wherein the linear guide sleeve is designed to guide the linear guiding device.

4. The apparatus according to claim 2, wherein the linear guiding device comprises a piston.

5. The apparatus according to claim 2, wherein the linear guiding device is movable mechanically, hydraulically, pneumatically, electrically or magnetically.

6. The apparatus according to claim 3, wherein a tension or pulling spring in the linear guide sleeve is arranged between the linear guiding device and the first anchor.

7. The apparatus according to claim 3, wherein the connection between the first anchor and the linear guiding sleeve is, or comprises a plug connection, a clamp connection, a bayonet lock or another connection.

8. The apparatus according to claim 1, wherein the pulling device or guiding device comprises an elongated, flexible and tensile element.

9. The apparatus according to claim 1, wherein the apparatus comprises a second anchor for implantation in or on the heart apex, a ligament, a rib, a sternum and/or a structure with local proximity to the heart apex.

10. The apparatus according to claim 1, wherein the apparatus comprises a metal, a plastic and/or a composite material.

11. A control device comprising a mechanical, hydraulic, pneumatic, electric or magnetic drive system for driving a pulling or guiding device of the apparatus according to claim 1.

12. An insertion system comprising:
an apparatus having a guiding device for displacing a first anchor; and
at least one of an insertion catheter, a guiding catheter, a guidewire and at least one delivery or supply catheter.

13. A kit comprising:
the apparatus according to claim 1,
a control device comprising a mechanical, hydraulic, pneumatic, electric or magnetic drive system for driving a pulling or guiding device, and/or
an insertion system having at least one of an insertion catheter, a guiding catheter, a guidewire and at least one delivery or supply catheter.

14. A method for supporting the heart action, said method comprising:
providing the apparatus according to claim 1;
implanting the first anchor in or on the heart base, the heart skeleton, the aortic root and/or a structure in local proximity to the aortic root;
implanting the pulling or guidance device for moving the first anchor; and
connecting the first anchor and the pulling or guiding device.

15. The method according to claim 14, further comprising:
implanting the second anchor in or on the heart apex, a ligament, a rib, a sternum and/or a structure in local proximity to the heart apex; and
connecting the first anchor and the second anchor.

16. The method according to claim 14, further comprising:
providing a control device comprising a mechanical, hydraulic, pneumatic, electric or magnetic drive system for driving the pulling or guiding device of the apparatus according to claim 1;
moving the pulling or guiding device using the control unit to support the heart action.

17. The apparatus of claim 3, wherein the first anchor is connected with or by at least two linear guide brushes to the at least one end of the first anchor.

18. The apparatus according to claim 8, wherein the elongated, flexible and tensile element is a rope or a belt.

19. The method of claim 14, further comprising an insertion system—having at least one of an insertion catheter, a guiding catheter, a guidewire and at least one delivery or supply catheter, wherein the insertion system is used during the implanting of the first anchor, the implanting of the pulling or guidance device for moving the first anchor, and the connecting of the first anchor and the pulling or guiding device.

20. The method of claim 15, wherein the insertion system is used during the connecting of the first anchor and the second anchor.
